(19) United States
(12) Patent Application Publication
(10) Pub. No.: US 2023/0326600 A1
(43) Pub. Date: Oct. 12, 2023

(54) A METHOD FOR DETERMINING A DIAGNOSTIC OUTCOME

(71) Applicant: IMPERIAL COLLEGE INNOVATIONS LIMITED, London (GB)

(72) Inventors: Ivana PENNISI, London (GB); Jesus RODRIGUEZ MANZANO, London (GB); Nicholaos MISCOURIDES, London (GB); Nicolas MOSER, London (GB); Dominic HABGOOD-COOTE, London (GB); Myrsini KAFOROU, London (GB); Jethro A. HERBERG, London (GB); Michael LEVIN, London (GB); Pantelis GEORGIOU, London (GB); Ahmad MONIRI, London (GB)

(21) Appl. No.: 18/042,965

(22) PCT Filed: Aug. 24, 2021
(86) PCT No.: PCT/EP2021/073427

§ 371 Date: Feb. 24, 2023

(30) Foreign Application Priority Data
Aug. 24, 2020 (GR) 20200100509
Sep. 21, 2020 (GB) 2014819.3

(51) Int. Cl. G16H 50/20; G16B 25/10; G16B 40/00

(52) U.S. Cl. CPC G16H 50/20; G01N 27/4145; G16B 40/00; G16B 25/10

(57) ABSTRACT

The present application relates to processor-implemented methods for determining a diagnostic outcome comprising receiving signals generated by an array of electro-chemical sensors in solution with a biological sample from a subject, each sensor of the array of electro-chemical sensors configured to generate a signal in response to one or more amplification reactions occurring in the solution, the one or more amplification reactions being indicative of the presence of an RNA host-response; processing the signals received from the array of electro-chemical sensors; based on the processing, assigning at least one value to the sample, the at least one value being indicative of a likelihood of the diagnostic outcome; based on the at least one assigned value, determining the diagnostic outcome. The application also relates to a method for determining a diagnostic outcome, a system comprising an array of electro-chemical sensors and a processor, and a computer readable medium comprising computer executable instructions which cause the processor to implement the method.

This disclosure relates to determining a diagnostic outcome, and in particular to a method for determining a diagnostic outcome using the RNA host-response.

### BACKGROUND

There is an urgent need for fast, cheap, robust and quantitative methods of distinguishing between different types of infectious or inflammatory diseases. When infectious or inflammatory diseases cannot be diagnosed in a timely manner, subjects may receive unnecessary treatments or risk misdiagnosis.

There are three main classes of diagnostic methods for detection and identification of pathogens: classical microbiology techniques (such as microscopy and cultivation), protein-based (such as antigen-antibody interactions) and nucleic acid-based (such as sequencing, polymerase chain reaction and microarrays) methods. Of these, only nucleic acid-based methods have a quantitative rather that qualitative output. However, these techniques rely on lab-based instruments such as microarrays, RNA sequencing and qPCR and are also expensive and slow to perform.

Traditional methods of nucleic acid-based detection use optical mechanisms based on fluorescence labelling that require large and costly equipment. Typically, this equipment makes such techniques unsuitable for point-of-care diagnostics.

Polymerase chain reaction (PCR), is the most common method of nucleic acid-based detection, within which the DNA amplification is done in cycles. In each cycle, the number of DNA molecules is doubled until one of the reactants have been consumed. Each PCR cycle typically comprise three steps (denaturation, annealing and extension) and each of these steps occur at a particular temperature. PCR has an appealing property that the number of DNA molecules can be easily quantified (2N, where N is the number of cycles). However, the disadvantage of PCR is that the temperature of the reaction must be controlled precisely; usually requiring a thermocycler. Ergo, PCR is unsuitable for use in point-of-care applications.

Whole genome sequencing is another method of nucleic acid-based analysis. This is primarily a research tool as it cannot be targeted, for example, to the detection of infectious diseases. It provides the raw nucleotide sequence of an individual's DNA and this large amount of data must be stored and analyzed. The time taken to receive a result would be detrimental to using this technique as a point-of-care test.

In summary, current nucleic acid-based detection methods, such as fluorescence-based techniques, are expensive, non-portable due to the need for precise temperature regulation and heavy optical equipment, do not provide spatio-temporal information, cannot be miniaturized into a small form factor device, and require a technically trained operator.

The present disclosure seeks to address these and other disadvantages encountered in the prior art by providing an affordable solution for quantitative detection of RNA-host transcripts highly expressed in the presence of specific diseases. The label-free detection method based on scalable microchip semiconductor technology combined with isothermal amplification chemistries makes this technology suitable to implementation at low cost. It can be delivered at the point-of-care without the need for large and expensive pieces of optical equipment in a lab, while still delivering a quantitative result that can lead to a diagnostic outcome.

### SUMMARY

According to an aspect of the present disclosure, there is provided a processor-implemented method for determining a diagnostic outcome. The method comprises receiving signals which are generated by an array of electro-chemical sensors in solution with a biological sample from a subject. Each sensor of the array of electro-chemical sensors is configured to generate a signal in response to one or more amplification reactions occurring in the solution where the one or more amplification reactions are indicative of the presence of an RNA host-response. The signals received from the array of electro-chemical sensors are processed. Based on the processing, at least one value is assigned to the sample, the at least one value being indicative of a likelihood of the diagnostic outcome. Based on the at least one assigned value, the diagnostic outcome is determined.

Optionally, the electro-chemical sensors are any of ion-sensitive field-effect transistor (ISFET) sensors, pH sensors, or chemically sensitive sensors.

Optionally, the nucleic acid amplification reaction is an isothermal reaction. Further, the amplification reaction may be a LAMP reaction.

Optionally, the biological sample contains at least one of a DNA, RNA or protein sample.

Optionally, the at least one value is assigned to the sample based on a gene expression value and/or time-to-positive.

The diagnostic outcome may be a differentiation between a bacterial and a viral infection. The diagnostic outcome may be a differentiation between a biological sample from a subject that has an infection and a biological sample from a subject that does not have an infection. Further, the subject that does not have an infection may instead has an alternative diagnosis. This alternative diagnosis may be an inflammatory illness. The diagnostic outcome may be a differentiation between a biological sample from a subject that is predisposed to an infection and a biological sample from a subject that is not predisposed to an infection. The diagnostic outcome may be a differentiation between an active form of an infection and a latent form of an infection.

Optionally, assigning the at least one value to the sample comprises using a trained classifier.

Optionally, the RNA host-response is detected using gene expression levels of a gene signature comprising one or more genes, selected from the group consisting of: IF144L, EMR1, FAM89A, EB13, IF127L, IFTI1, RSAD2, IFIT3, OTOF, IFIT2, EPSTI1, SERPING1, OAS1, IF16, HLA-DRB6, HBZ, HS.386275, EIF2AK2, IFIT1L, FCER1A, C210RF7, GYPE, GYPB, HBM, EIF1AY, LOC649143, HBD, FBXO7, KCNMA1, MERTK, UPB1, PTPN20, TMEM119, SLPI, S100P and P13.

Optionally, processing the signals may comprise clustering the signals received from each sensor of the array of sensors in the time domain. Further, each cluster generated by the clustering process may comprise a subset of sensors of the array of sensors.

Optionally, processing the signals may comprise obtaining a feature associated with the amplification curve associated with an amplification reaction. The amplification curve describes a degree of amplification over time. The at least one value may be assigned based on the feature associated with the amplification curve. Further, the feature associated with the amplification curve may be one of a time-to-positive and a Ct value.

Optionally, processing the signals may comprise obtaining a feature associated with a plurality of different amplification curves, each amplification curve being associated with a different amplification reaction and each describing a degree of amplification of a respective amplicon over time. The at least one value may be assigned based on the plurality of features associated with the amplification curve.

According to another aspect of the present disclosure, there is provided a method for determining a diagnostic outcome. The method comprises placing a biological sample from a subject in solution with an array of electro-chemical sensors. Each sensor of the array of electro-chemical sensors is configured to generate a signal in response to one or more amplification reactions occurring in the solution, the one or more amplification reactions being indicative of the presence of an RNA host-response. The signals received from the array of electro-chemical sensors are processed. Based on the processing, at least one value is assigned to the sample according to the likelihood of a diagnostic outcome. Based on the at least one assigned value, the diagnostic outcome is determined.

According to another aspect of the present disclosure, there is provided a system comprising an array of electro-chemical sensors and a processor. Each sensor of the array of electro-chemical sensors is configured to generate a signal in response to one or more amplification reactions occurring in the solution, the one or more amplification reactions being indicative of the presence of an RNA host-response. The processor is configured to process signals received from the array of electro-chemical sensors. Based on the processing, at least one value is assigned to the sample, the at least one value being indicative of the likelihood of a diagnostic outcome. Based on the at least one assigned value, a diagnostic outcome is determined.

According to another aspect of the present disclosure, there is provided a computer readable medium comprising computer executable instructions which, when executed by a processor, cause the processor to perform implementations of the disclosed methods.

### FIGURES

Specific embodiments are now described, by way of example only, with reference to the drawings, in which:

FIG. 1 depicts a method according to the present disclosure.

FIG. 2 depicts a diagnostic workflow.

FIG. 3 depicts an example workflow of assay design.

FIG. 4 depicts a schematic diagram depicting a typical configuration of an ISFET sensor. In particular, a cross-section diagram of the ISFET structure in commercial CMOS (complementary metal-oxide-semiconductor) technology is depicted.

FIG. 5 depicts a workflow for processing raw data from the array of sensors in order to find a time-to-positive.

FIG. 6 depicts a sample classifier consisting of a supervised machine learning algorithm and a 2-gene signature (IF144L and EMR1).

FIGS. 7a and 7b show the 2-transcript signature applied to children with bacterial and viral infections using microarray data

FIGS. 8a and 8b shows the 2-gene signature translated to RT-eLAMP.

FIG. 9 is the abundance of IF144L using RNASeq.

FIG. 10 depicts the correlation between length and depth normalised exon counts for IF144L, where primer spans an exon-exon boundary between highlighted exons.

FIG. 11 is the abundance of EMR1 using RNASeq.

FIG. 12 depicts the correlation between length and depth normalised exon counts for EMR1-ADGRE1, where primer spans an exon-exon boundary between highlighted exons.

FIG. 13 illustrates a block diagram of one implementation of a computer device.

### DETAILED DESCRIPTION

The present application relates to a method of determining a diagnostic outcome. It comprises placing a biological sample from a subject in solution with an array of electro-chemical sensors. Each sensor of the array is configured to generate a signal in response to amplification reactions occurring in the solution, indicative of the presence of an RNA host-response. The signals from the array of sensors are processed, and based on the processing, at least one value is assigned to the sample. Based on the at least one assigned value, a diagnostic outcome is determined.

According to an aspect, the value assigned to the sample reflects the likelihood of the sample having a certain diagnostic outcome. It may be based on any feature of the amplification curve, and interactions between values derived from different reactions. For example, the Ct value of gene A, gene B or the difference between gene A and gene B are all valid values. It may also be based on the entire amplification curve. The value may be based on the gene expression values or time-to-positive values from signals of the array of sensors.

According to an aspect, the diagnostic outcome may be one of: differentiating between a bacterial and a viral infection; differentiating between an infected sample and a non-infected sample; differentiating between the latent and active form of an infection; differentiating between a sample predisposed to an infection and a sample that is not predisposed to an infection.

Overview

The present disclosure relates to devices and methods that can be used in some embodiments to return a diagnostic outcome from analysis of a biological sample.

According to a specific implementation, the application relates to a method of classifying RNA host-response signatures using microchip technology.

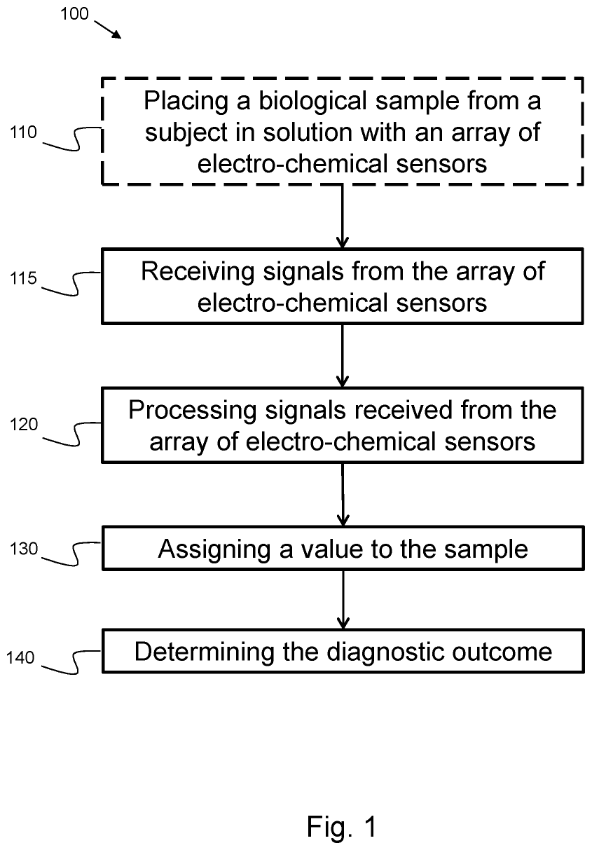

FIG. 1 depicts a workflow according to the present disclosure. Blocks 115 to 140 may be performed by a computer. At block 110, a biological sample from a subject is placed in solution with an array of electro-chemical sensors. The subject may be an individual, such as a patient. While reference is made to a ‘patient’ this does not imply that the individual is necessarily ill, infected, or suffering from ill-health. The present techniques are applicable to healthy individuals. Each sensor of the array is configured to generate a signal in response to amplification reactions occurring in the solution, indicative of the presence of an RNA host-response. At block 115, the generated signals are received by a processor.

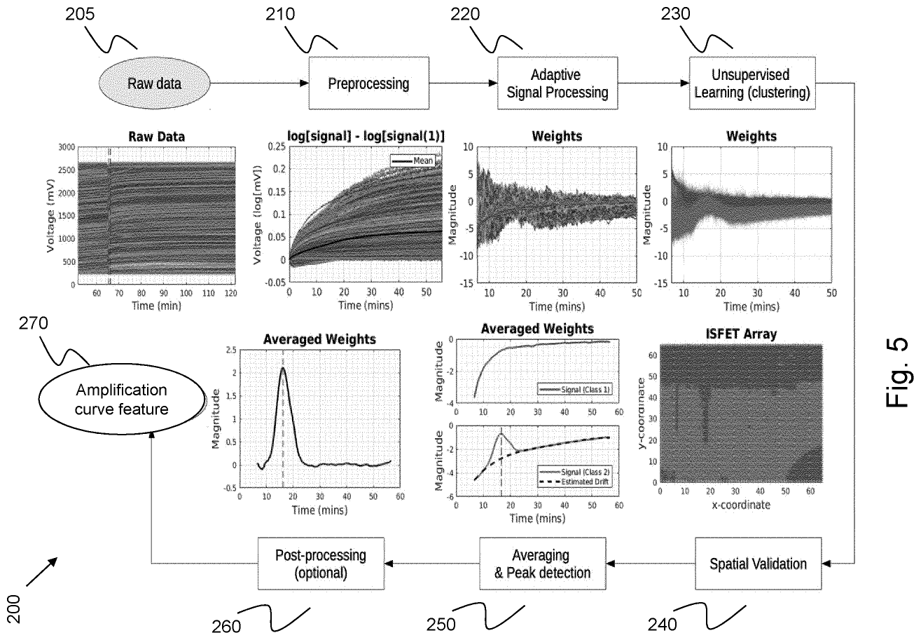

At block 120, the signals from the array of sensors are processed. A suitable workflow for block 120 is depicted in FIG. 5. By virtue of processing the signals, a feature associated with the amplification curve of a particular amplicon may be obtained. The amplification curve may take the form of a time series depicting the degree of amplification of a particular amplicon over time. In particular, the amplicon is RNA associated with the presence of a host-response. As will be described later, the feature of the amplification curve may be a time to positive, a Ct value, or may be the shape of the amplification curve or a peak in a data series produced by an incremental learning algorithm.

Optionally, signals received from multiple different reactions may be received at block 115 and processed at block 120 such that features of multiple different amplification curves are obtained. Each amplification curve is associated with a different amplicon.

At block 130, based on the processing, at least one value is assigned to the sample. The value is indicative of a likelihood of a particular diagnostic outcome. As will be described later, the at least one value may be a score, e.g. a signature score. The diagnostic outcome may be a differentiation between a bacterial and a viral infection; or else may be a differentiation between a biological sample from a subject that is infected and a biological sample from a sample that is not infected.

The value assigned to the sample reflects the likelihood of the sample being associated with a certain diagnostic outcome. The value may be assigned based on features of the amplification curve for one or more amplicons. For example, the value may be assigned based on interactions between values derived from different reactions, where the different reactions are each associated with a different amplicon. For example, the Ct value of gene A, gene B or the difference between gene A and gene B may all be used in order to determine. It may also be based on the entire amplification curve. The value may be based on the gene expression values or time-to-positive values from signals of the array of sensors.

The assigning of a value may be performed by a classifier. The purpose of the classifier is to output a value, e.g. based on inputted amplification curve feature(s), which describes a likelihood of a particular diagnostic outcome. The value may be a percentage likelihood, for example, of the diagnostic outcome.

The at least one value may be assigned using statistical classification techniques and/or machine learning techniques. For example, the at least one value may be assigned using a trained classifier. In this case, the at least one value is the output of the trained classifier. The trained classifier may be trained by using samples taken patients who are known to have particular infections, and from individuals known to not have the particular infection. By processing the signals, e.g. in the manner depicted in FIG. 5, a labelled data set may be generated. This data set may be used to train the classifier in a known way according to supervised machine learning principles.

FIG. 5 is a workflow in which a single amplification curve feature associated with a single amplicon is produced. This amplification curve feature may be inputted into the classifier, either alone or in combination with other amplification curve features. According to other implementations of the present disclosure, blocks 205 to 270 are performed multiple times, each in connection with a different amplification reaction, in order to generate amplification curves associated with a plurality of different amplicons. This plurality of amplification curve features may then be passed to the classifier. In this way, gene signatures comprising of multiple genes can be analysed. The trained classifier may take, as an input, the Ct value of gene A and the Ct value of gene B; or else the difference in these values between gene A and gene B, or other values based on the plurality of amplification curve features.

At block 140, based on the at least one assigned value, the diagnostic outcome is determined. This determination may occur according to a threshold certainty value, for example if the value assigned to the sample at block 130 indicates at least a 90% likelihood of a diagnostic outcome, then that diagnostic outcome is determined at block 140. For example, the classifier may output a value which indicates that the subject has a viral, rather than a bacterial, infection with a certainty of 95%. As this degree of certainty is above the threshold certainty of 90%, it is determined that the subject has a viral infection.

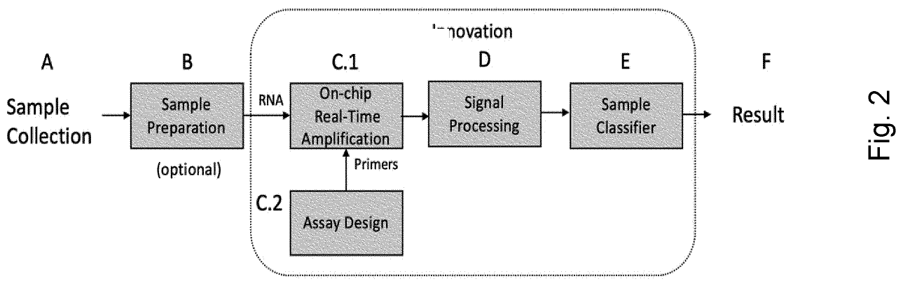

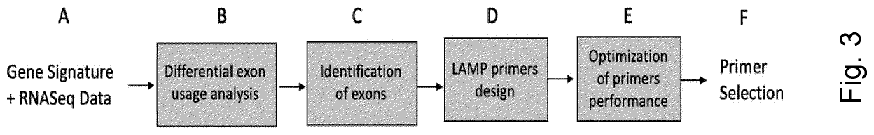

FIG. 2 depicts a diagnostic workflow. Step A is sample collection and step B is an optional step of sample preparation, for example to extract RNA from whole blood. At step C, there is real time amplification. C1 is the assay design (shown in more detail in FIG. 3) and C2 is microchip-based amplification and sensing. Step D is signal processing, which may comprise extracting a time-to-positive. Step E is the application of an algorithm to classify the diagnostic outcome. The host blood can then be assigned an RNA signature score and a classification result, for example whether the sample has a bacterial or a viral infection.

FIG. 3 depicts an example workflow of assay design. At step A, the inputs may be the gene signature and RNA-Seq data. At step B, differential exon analysis is carried out in order to detect high variance in expression of single or multiple exons. At step C, the area under the curve is used to identify exons with high and discriminating expression levels. Step D is specific design of 4-6 LAMP primers targeting 6-8 sequence regions capturing mature RNA. Step E is optimisation of primers based on chemistry performance. The output is then the primer selection. The gene signature may be split into on-chip amplification in several ways. For example, in the case of a 4-gene signature (A, B, C, D), four separate reactions may be run or two reactions may be run, one including primers for A+B and the other for C+D.

ISFET

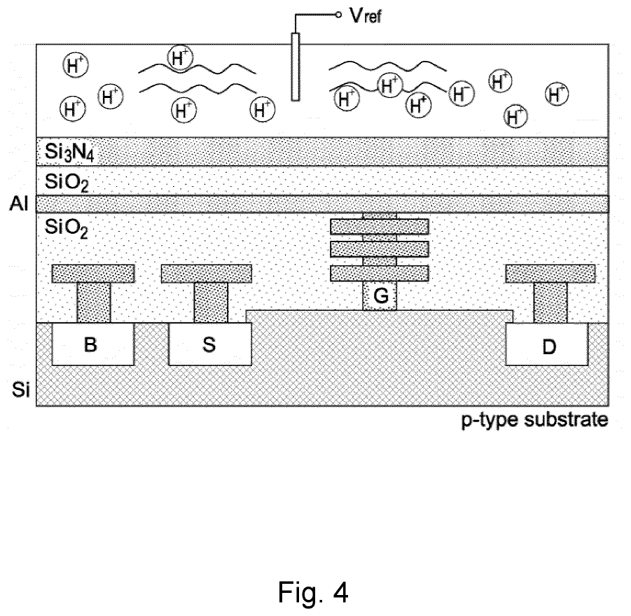

FIG. 4 is a schematic of an ISFET. The ISFET shares features and functionality with the well-known MOSFET and comprises a source, a gate, a body, a channel, and a drain. Charge carriers (e.g. electrons or holes) enter the channel at the source and exit the channel via the drain. The MOSFET works by electronically varying the effective width of the channel along which charge carriers flow by altering the voltage at the gate. This allows the voltage and current flow between the source and drain to be controlled. The voltage at the gate which is required in order for the channel to conduct charge carriers between the source and drain is called the threshold voltage. The functionality of MOSFETs will be well known to the skilled person and will not be discussed further.

The ISFET works in a similar manner to the MOSFET, and also comprises a source, drain, body, and gate. In comparison with the MOSFET, the ISFET can be modified into a biosensor by replacing the gate with a membrane in contact with a chemical solution. In this manner the number of charge carriers, i.e. ions, in the solution directly affects the device threshold voltage. The ISFET can be tailored to detect, or sense, particular chemicals and/or ions by depositing an ion-selective membrane on the gate. Insulators, such as Aluminium Oxide (Al₂O₃), Silicon Nitride (Si₃N₄), Hafnium Oxide (HfO₂), Tantalum Pentoxide (Ta₂O₅) and Silicon Dioxide (SiO₂), make the transistor sensitive to pH, and therefore, this makes the ISFET useful for DNA detection. The skilled person would appreciate that ISFET sensors used in the present application may take a variety of forms and configurations, and may be fabricated with negative-channel or positive-channel MOS technology. The ISFET sensors may be doped and may be of the PMOS or NMOS type.

Given that the gate of the ISFET is essentially made of the chemical solution in contact with the insulator and biased using a reference electrode, the threshold voltage of the ISFET will be sensitive to pH fluctuations, i.e. the number of protons released. Furthermore, the threshold voltage can be measured using analogue circuitry, and thus by measuring changes in the threshold voltage of an appropriately configured ISFET it is possible to detect the presence of specific ions in the solution.

Generally, the ISFET sensors may be configured to measure the pH of an electrolyte (i.e. the H+ ion content of the electrolyte), but they can be made sensitive to ions other than H⁺ through the choice of the ion-sensitive membrane (such as Mg²⁺, Ca²⁺, Na⁺ or K⁺), thus adding an element of ion-selectivity. Unlike conventional fluorescent-based nucleic acid analysis systems, an Ion-FET based platform does not require expensive optical instruments or radioactive isotopes for detection, thus making the platform of the present disclosure a cost effective, safe and simple alternative for sensing molecules.

As discussed previously, there are several disadvantages of current nucleic acid-detection methods such as PCR. Nucleic acid-detection can be performed by running the LAMP reaction on an ISFET array in order to overcome these issues. Firstly, there is no need for a thermocycler, allowing the diagnostic platform to be portable. Secondly, there is no need for expensive optical machinery to measure fluorescence given that the ISFET array is sensitive to pH. The fact that LAMP reactions have a considerable pH change means that a detectable signal is produced from nucleic-acid amplification.

Presently disclosed methods combine the use of isothermal nucleic acid amplification reaction techniques with semiconductor-based technology, resulting in the best properties of current methods: cheap, robust, quantitative and suitable for point of care.

Signal Processing

FIG. 5 is a workflow for processing raw data received from the array of sensors. Some or all of the workflow depicted at FIG. 5 may be employed in block 120 of FIG. 1.

At block 205, raw data is received from the array of electro-chemical sensors. The raw data comprises signals generated by the array of electro-chemical sensors. The electro-chemical sensors may be ion-sensitive field-effect transistor (ISFET) sensors, or pH sensors or other forms of chemically sensitive sensors. The array of sensors is positioned in solution with a biological sample from a subject, such as a patient. is Each sensor of the array of electro-chemical sensors is configured to generate a signal in response to one or more amplification reactions occurring in the solution. The assay/solution is designed such that amplification occurs in response to the presence of a host response in the biological sample. The one or more amplification reactions are therefore indicative of the presence of an RNA host-response.

Together, the signals take the form of a time series. This is depicted in the graph which accompanies block 205. The graph shows multiple signals received from each sensor of the array of sensors. Each line on the graph depicts signals received from a particular sensor of the array of sensors. Along the x axis is time, measured in minutes. On the y-axis is voltage measured in log (mV). The signals are indicative of amplification having occurred in solution, which need not be measured in volts but instead may be measured according to a degree of fluorescence or pH in a known manner.

By way of an example, we may consider that the sample has been taken from a patient suffering from an infection, and it is desirable to determine whether the patient's infection is viral or bacterial in nature. In other words, the aim is to discriminate between a viral and a bacterial infection. When the patient was infected, and thereby when the cells of the patient's body were exposed to the infection, a host response occurs. The host response may be described as the reaction of the patient's body to the infection. As part of the host response in this example, RNA is generated. By identifying and classifying the RNA generated as part of the host response, it is possible to determine whether the infection is viral or bacterial. The assay/solution is designed and prepared such that amplification occurs in the presence of a particular RNA, which itself is indicative of the presence of the host response.

Block 210 is an optional pre-processing step. Any of background subtraction or normalization may occur. Each signal may be normalised.

At block 220, adaptive signal processing is carried out on the data by assigning a weight to each signal. This ‘adaptive signal processing’ may be referred to herein as ‘online learning’ or ‘incremental learning’. Adaptive signal processing techniques are is examples of machine learning. The signals received from the array of sensors are fed into a model. The received signals are fed into the model as a time series and are used to continuously extend the existing model's knowledge. In other words, the received signals are used to train the model. The weights assigned to each signal varies with time. The aim of the model is to predict the future value of the signals based on historical data. Therefore, if the weight changes through time, it suggests there has been some change in the underlying data, i.e. in the received signal. Accordingly, adaptive signal processing techniques may be used to detect changes in behaviour in the signals. The graph which accompanies block 220 depicts the change in the magnitude of each received signal with time.

At block 230, the weighted signals are clustered into those sensors that have detected an amplification reaction in the solution and those sensors that have not. An unsupervised machine learning model may be used to cluster the signals. In a specific example, a k-means clustering algorithm may be employed, though any suitable clustering technique may be used. Here, the clustering occurs in the time domain. The graph which accompanies block 230 depicts the change in the magnitude of each received signal with time, where the signals have been clustered (i.e. grouped) in the time domain.

At block 240, a clustering validation technique is employed. Multiple different clustering techniques may be employed. In an example of an external clustering validation process, the clustering can be verified based on the location of the clustered sensors in the array of sensors. Each signal in the time series received at block 205 is associated with a particular sensor, which has a particular location in the array of sensors. In other words, each received signal may be labelled with the location of the sensor in the array. By converting the signals from temporal to spatial form, the resulting clustering can be analysed to determine if the clustering at block 230 is valid. It may be expected that those electro-chemical sensors that have detected amplification reactions will be close to each other in the array. That is, it is expected that sensors close to each other will exhibit similar behaviour.

The graph which accompanies block 240 depicts the x and y co-ordinates of each sensor in the array (in this example the array is an ISFET array). It can be appreciated by inspection that the clustering in the time domain at block 230 is valid, as the clusters exhibit a good degree of spatial dependence in the figure. This validation stage may be manual, or else may be automatic and/or performed by an algorithm or other processor-implemented method.

At block 250, the weights of each cluster are averaged. A peak is to be expected in the signals received from each cluster of sensors that detected an amplification reaction. The peak is identified using any of a number of known techniques for peak detection. Peak detection is a common activity in signal processing and the skilled person will be familiar with methods of peak detection in time series data.

Block 260 is an optional post-processing step. The peak detected at block 250 may be used to find the time-to-positive. The time-to-positive may be defined as the time at which the peak occurs, in an implementation in which the received signals form a time series. As discussed above, as a result of the adaptive signal processing at block 220 and the clustering at block 230, the peak indicates a time at which the behaviour of the received signals changed most dramatically during the amplification reaction. The ‘time to positive’, tp, may therefore be regarded as the time from the beginning of the reaction until a positive determination that the DNA is amplifying. In examples, the time to positive may be taken as the time for half of the amplification to complete. The time-to-positive may be used to determine the value assigned to the biological sample which is used to determine the diagnostic outcome.

While reference is made to a time series measured in minutes, the time series may be instead tracked by the number of cycles in a PCT reaction. In such an implementation, the peak may occur at a Ct value, or threshold cycle value. This value is the cycle number at which a positive determination can be made that the RNA is amplifying. For example, the threshold cycle number may occur when the fluorescence generated within a reaction crosses a fluorescence threshold, a fluorescent signal significantly above the background fluorescence. At the threshold cycle, a detectable amount of amplicon product has been generated during the early exponential phase of the reaction.

At block 270, a feature of the amplification curve is obtained. As discussed above, this may be the time to positive, or a Ct value. The feature of the amplification curve obtained at 270 may be the shape of the peak detected at block 250, for example its height and/or standard deviation. The feature of the amplification curve may be the shape of the entire amplification curve. Alternatively, multiple such features may be obtained. These features may be used, individually or in combination, by a classifier in order to determine which gene is present in solution.

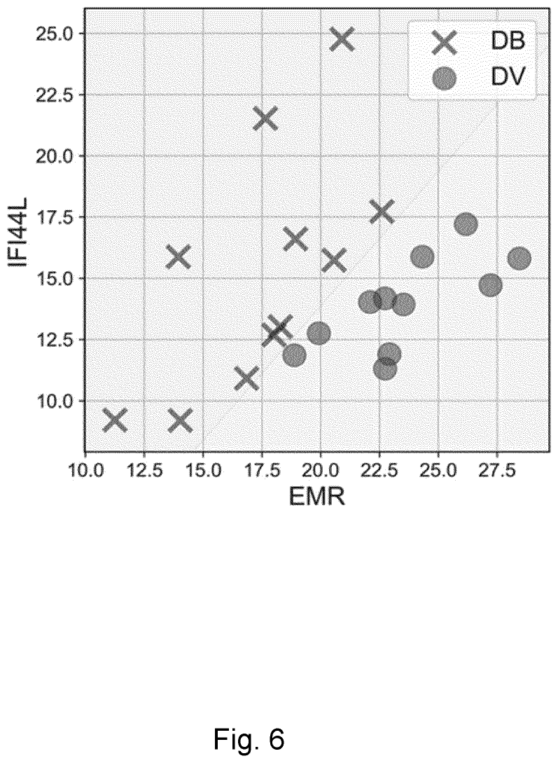

It will be appreciated form the above description that the workflow of FIG. 6 may be used to obtain a feature of an amplification curve associated with a particular gene, which itself is indicative of the host-response. In order to obtain such features for each gene forming a gene signature, the gene signature can be split into on-chip amplifications in several ways. For example, for a 4-gene signature (A,B,C,D), it is possible to either run 4 separate reactions (i.e. put a biological sample in 4 different solutions, each containing an array of electro-chemical sensors), or run 2 reactions where 1 reaction includes primers for A+B and the other has C+D.

At block 280, the amplification curve feature(s) is passed to a classifier. The purpose of the classifier is to output a value, based on the inputted amplification curve feature(s), which describes a likelihood of a particular diagnostic outcome. The value may be a percentage likelihood, for example, of the diagnostic outcome.

The at least one value may be assigned using statistical classification techniques and/or machine learning techniques. For example, the at least one value may be assigned using a trained classifier. In this case, the at least one value is the output of the trained classifier. The trained classifier may be trained by using samples taken patients who are known to have particular infections, and from individuals known to not have the particular infection. By processing the signals, e.g. in the manner depicted in FIG. 5, a labelled data set may be generated. This data set may be used to train the classifier in a known way according to supervised machine learning principles.

According to implementations of the present disclosure, blocks 205 to 270 are performed multiple times, each in connection with a different amplification reaction, in order to generate amplification curves associated with a plurality of different amplicons. This plurality of amplification curve features may then be passed to the classifier. In this way, gene signatures comprising of multiple genes can be analysed. The trained classifier may take, as an input, the Ct value of gene A and the Ct value of gene B; or else the difference in these values between gene A and gene B, or other values based on the plurality of amplification curve features.

FIG. 6 depicts a sample classifier consisting of a supervised machine learning algorithm and a 2-gene signature (IF144L and EMR1). DB refers to a definite bacterial sample and DV to a definite viral sample. The x-axis is the time to positive value in minutes for EMR1 and the y-axis is the time to positive value in minutes for IF144L.

Bacterial Vs. Viral

### INTRODUCTION

Discrimination of viral from bacterial infections remains a challenge, resulting in unnecessary investigation, admission and antibiotic treatment of many febrile subjects. It is known that children with bacterial and viral infection can be distinguished by their blood host RNA signature. Here, it is demonstrated that a 2-gene RNA signature can be translated into a rapid (<25 minutes), and portable lab-on-a-chip platform suitable for development as a point-of-care test.

Methods

Herberg and colleagues reported a 2-transcript signature (IF144L and FAM89A) discovered using gene expression microarrays in a set of 455 febrile children with bacterial and viral infections (IRIS study, 09/H0712/58). 24 RNA samples were randomly selected from subjects in the IRIS study1 (St Mary's Research Ethics Committee approval 09/H0712/58) with confirmed bacterial (n=12) and viral (n=12) infections, matched for severity and collected between 2009-2017 and extracted using the PAXgene Blood RNA Kit (©PreAnalytiX GmbH). Suitable transcripts were identified for translation to our lab-on-chip platform, which uses Reverse Transcription Loop-mediated Isothermal Amplification (RT-LAMP), by assessing counts of IF144L and FAM89A in a publicly available blood RNA-Seq dataset comprising 255 children with bacterial and viral infections (GSE69529). The average gene counts for IF144L were sufficient (2281.9), but low for FAM89A (20.2), potentially compromising transferability across platforms. Therefore, using the list of previously identified 38 highly correlated transcripts, FAM89A was replaced with EMR1-ADGRE1 (RefSeq ID: NM_001974.5), which had sufficient average gene counts (511.3)4, and which in combination with IF144L (RefSeq ID: NM_006820) had a similar performance to the original 2 transcript signature (area under the curve (AUC) in the training, test and validation datasets 93.4%, 97.4%, 97.2% respectively).

The lab-on-chip platform combines novel pH-sensing complementary metal-oxide semiconductor (CMOS) technology5 with RT-LAMP, which we term electronic RT-LAMP (RT-eLAMP). RT-eLAMP uses thousands of microsensors (Ion-Sensitive Field-Effect Transistors) to detect H+ ions released, resulting in a change in pH, during the nucleic acid amplification process following the same experimental conditions as reported in our previous paper.

Gene expression values (normalised log 2 fluorescence for microarrays1) or time-to-positive values were compared from signals of all the microsensors for RT-eLAMP) using support vector classifiers, with 10-fold cross-validation, and each subject was assigned a score reflecting risk of bacterial or viral disease (package: scikit-learn in Python). The predictive accuracy of the score in subjects with microbiologically confirmed diagnoses was evaluated using receiver operating characteristic (ROC) curves, AUC, and the 95% confidence intervals (CI) under the binomial distribution.

Results

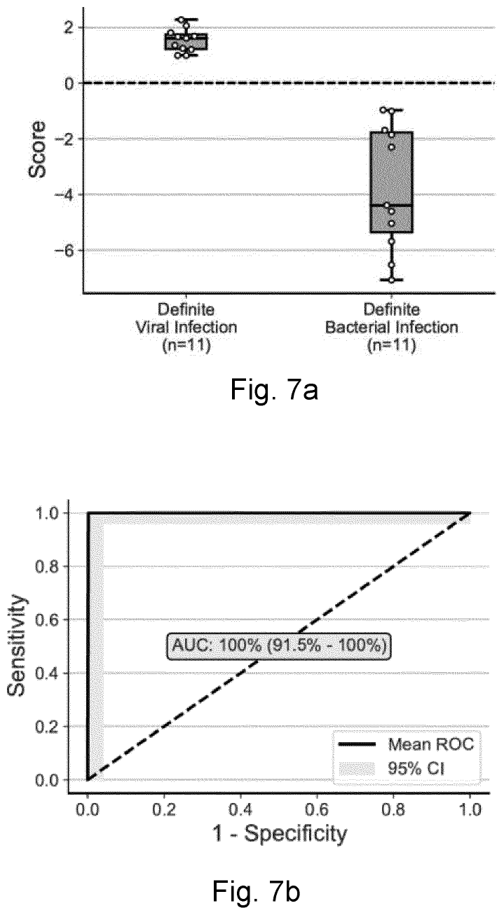

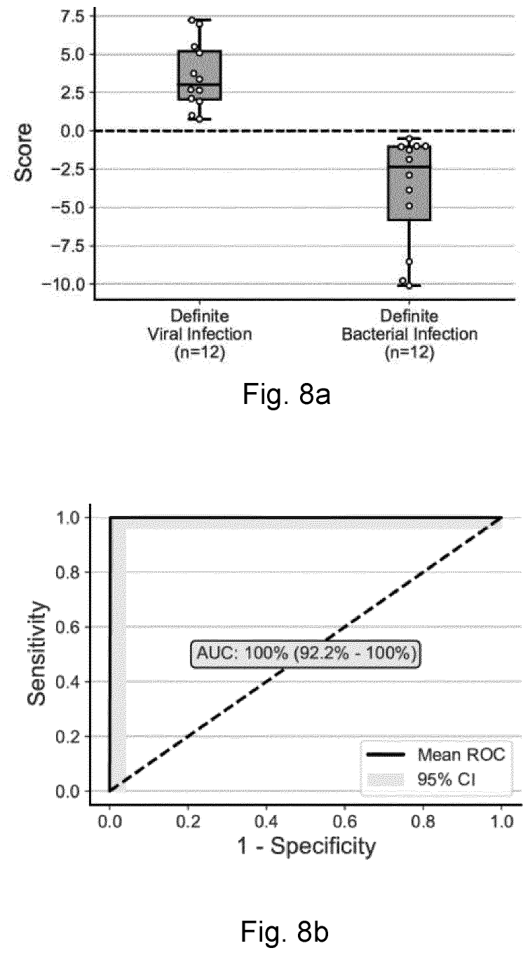

Using microarray data, the 2-transcript signature applied to children with bacterial and viral infections had a sensitivity and specificity respectively of 100% (95% CI, 95.7%-100%), and 100% (95% CI, 95.7%-100%), with AUC 100% (95% CI, 91.5%-100%) (FIGS. 7a and 7b). Translating the 2-gene signature to RT-eLAMP showed similar results: sensitivity and specificity were 100% (95% CI, 96.0%-100%), and 100% (95% CI, 96.0%-100%), with AUC 100% (95% CI, 92.2%-100%) (FIGS. 8a and 8b). The calculated limit-of-detection for the 2 assays were 10 RNA copies/reaction in 10 μL reaction volume and a time-to-positive under 25 minutes, much faster than microarray.

Discussion

Previously reported RNA signatures that differentiate bacterial from viral infection were discovered using cumbersome transcriptomic technologies. The promising transcript signatures have been moved closer to clinical application by establishing that the 2-transcript signature can be detected using a semiconductor-based sensing platform combined with isothermal amplification chemistries. The absence of fluorescent labels and the economy of scale of the microchip industry makes the technology potentially suitable for implementation at low cost (<£1 per chip).

Additional Comments

In the description of the 2-transcript bacterial-viral classifier (Herberg JAMA 2016), the discovery and validation groups included a wide mix of subjects drawn from subjects presenting to hospital with a febrile illness that was of sufficient severity to justify clinically-indicated blood testing. This covered a broad spectrum of disease severity, in both bacterial and viral groups. The results presented confirmed that prediction of bacterial illness was independent of the severity of the illness amongst those taking part (Herberg JAMA 2016—eApendix). The above method has again been carried out on subjects with a range of severity—some with moderate and some with severe illness. Eight of 24 subjects required PICU admission for their treatment. A key advantage of a point-of-care diagnostic would be for its use in primary care settings, where the spectrum of disease would include those with a milder phenotype.

The correlation of the predictive score in the RT-eLAMP and traditional RT-qLAMP for each subject was assessed to judge the sensitivity of the new technology. The predictive scores were tightly correlated between the 2 platforms, the Pearson's correlation coefficient was 0.90, R² 0.82 and the F-statistic p-value 1.36e-09.

Splice Variants Analysis

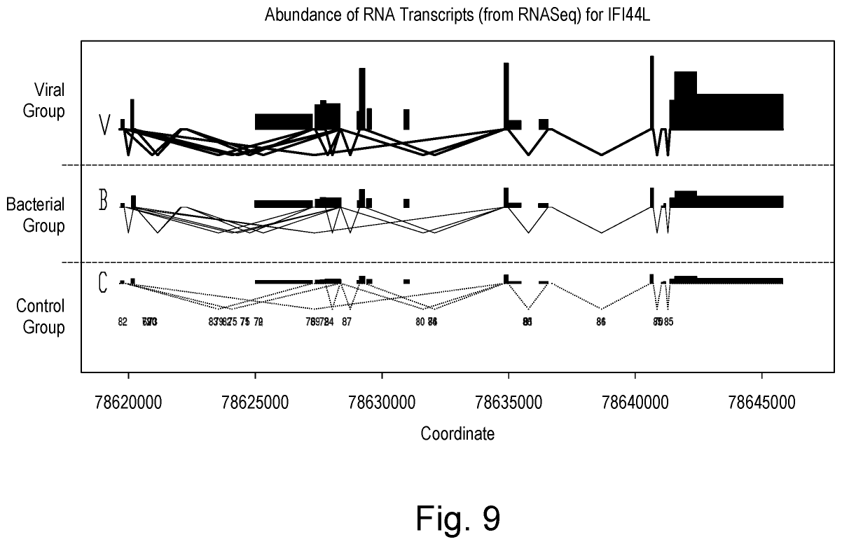

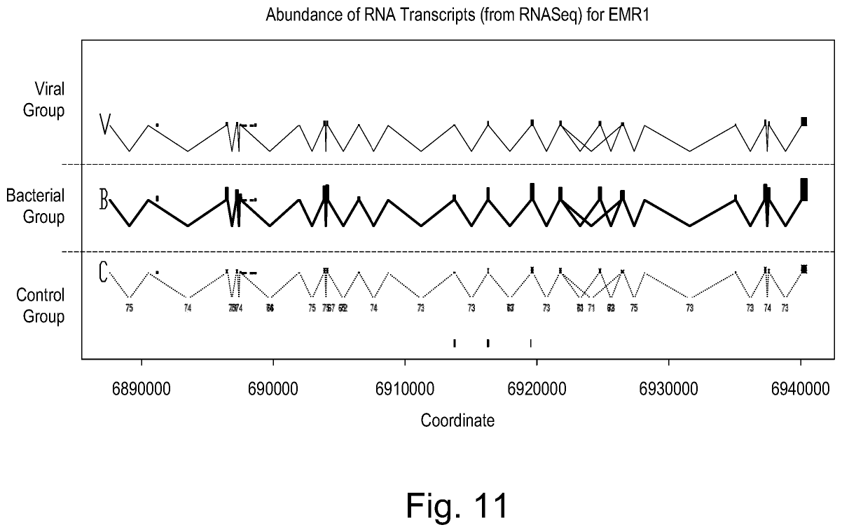

RNA-seq data can be used to investigate splice events of the two gene signature (respectively IF144L and EMR1-ADGRE1) aligned for each subject group RNA-seq reads (Bacterial, Viral and Healthy control) (FIGS. 9 and 11).

The height of the bar correlates with the abundance of the gene and this allows the exact location of the RNA transcript where the gene is highly expressed to be selected and can be clearly differentiated among different subject groups. The small beads labelled P in FIGS. 9 and 11 indicate the location where the primers sit for each of the gene of interest. They are spanning to exon therefore only capturing mature RNA, rather than genomic DNA.

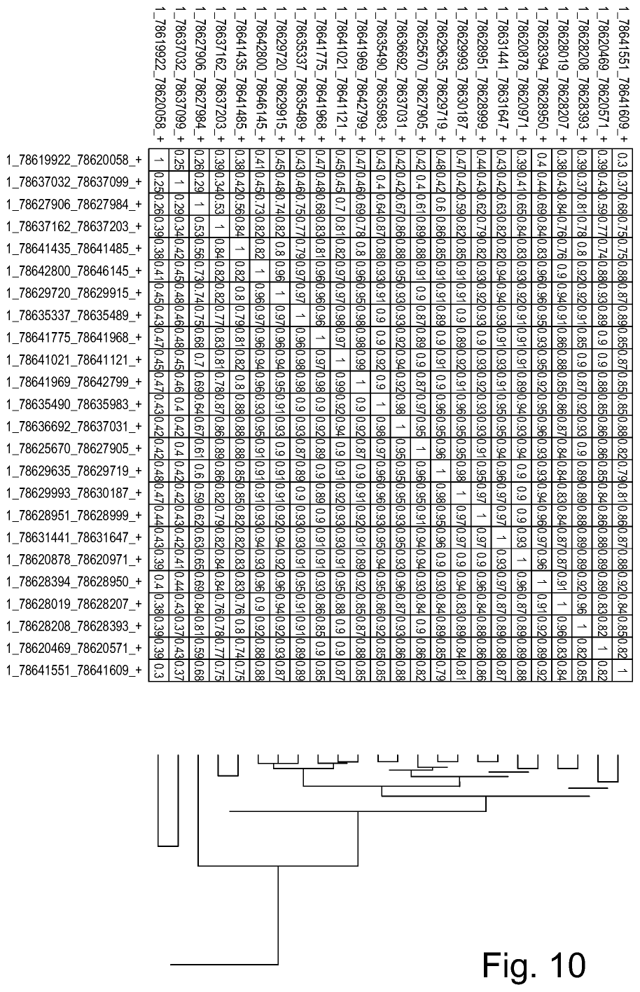

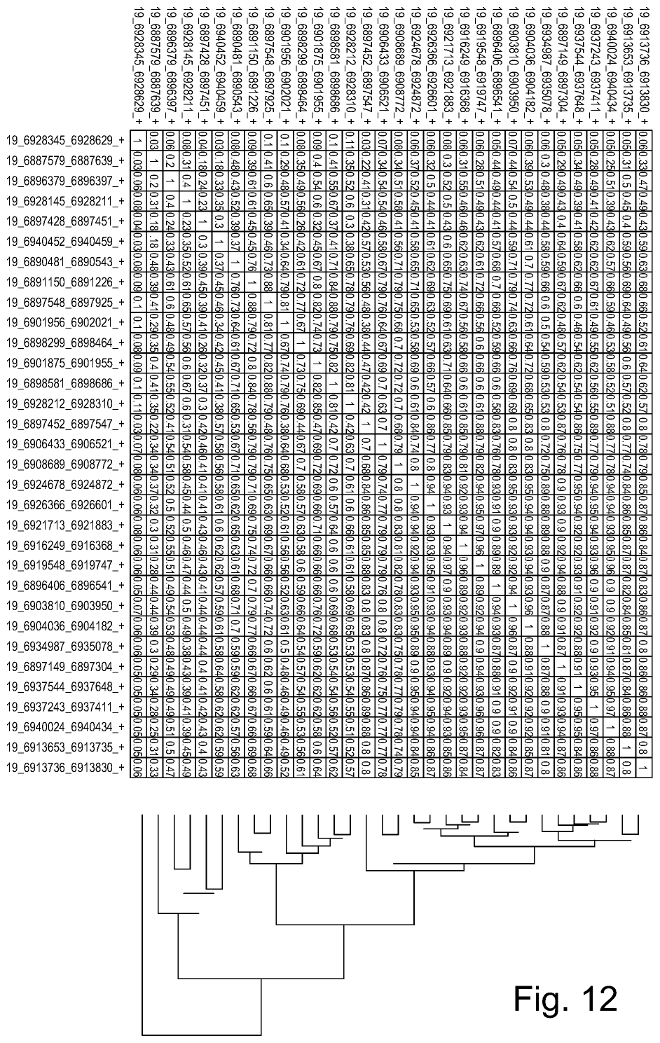

FIGS. 10 and 12 indicate the correlation between length and depth normalised exon counts for each of the 2-transcripts IF144L and EMR1-ADGRE1, where primer spans an exon-exon boundary between highlighted exons. All this information together provides a strong evidence that specific LAMP primers can be designed in the right location of the gene where the high expression of the transcript allows the detection of tiny amount of RNA, enabling a quantitative analysis to better discriminate among subjects group and providing a robust classification of bacterial and viral infections.

pH-LAMP Reagents

The existing isothermal amplification reagents used are not suitable for the detection of the pH change (detection of hydrogen ions released on chip surface), therefore the chemistry of the isothermal amplification was optimised in order to monitor the release of hydrogen ions through monitoring pH change in the solution due to amplification.

The optimised pH-LAMP reaction mix comprises: 1 μL of 10X isothermal customized buffer (pH 8.5-9), 0.6 μL of MgSO4 (100 mM stock), 0.56 μL of dNTPs (25 mM stock), 0.6 μL of BSA (20 mg/mL stock), 1.6 μL of Betaine (5 M stock), 0.042 μL of Bst 2.0 DNA polymerase (120,000 U/mL stock), 0.25 μL of NaOH (0.2 M stock), 1 μL of 10×LAMP primer mixture, 0.25 μL of AMV (25 U/μL stock, Promega), 0.10 μL of RNAse inhibitor(20 U/μL stock), 0.25 μL of SYTO 9 Green (20 μM stock),1 μL of RNA template and enough nuclease-free water (ThermoFisher Scientific) for a total volume of 10 μL/reaction.

Infected vs. Not Infected with SARS-CoV-2

### BACKGROUND

A reliable diagnostic point-of-care test that identifies subjects with bacterial or viral infection would have an immense impact on patient care. It would reduce unnecessary hospital admissions, invasive investigations and healthcare costs, and contribute to the reduction in antibiotic resistance by better regulating treatment with antibiotics. The approach in this disclosure, focusing on recognition of the distinct host transcriptomic responses underlying different infections, rather than identification of the pathogen, represents a paradigm shift in diagnosis.

The methods described herein can be applied to a diagnostic outcome of infection or non infection, for example with SARS-CoV-2. Diagnosis based on detection of the virus lacks sensitivity, does not identify subjects with inflammatory disease associated with previous SARS-CoV-2 infection, and does not exclude co-existence of other diseases with similar symptoms. There is an unmet need for accurate tests that can guide treatment decisions. It would be hugely beneficial to have cheap, accurate, geo-tagged point-of-care tests based on detection of the host gene expression response rather than pathogen. This test will guide diagnosis, treatment and surveillance in the ongoing COVID-19 pandemic, producing rapid public health impact. It does not necessarily rely on viral detection, and instead can exploit the differences in the host response.

Scientific Background

Previous work has identified that viral infections such as RSV and influenza, and inflammatory diseases such as Kawasaki Disease, have unique gene expression signatures, and that small signatures can be used for disease diagnosis—for example 2 transcripts distinguish bacterial and viral infection. A small whole blood transcript signature can distinguish COVID-19 from comparator infections, and identify inflammatory presentations including PIMS-TS. In adults, early viral, severe pulmonary and post-viral inflammatory presentations may be contiguous or overlapping, and progression is unpredictable. In children, overt viral disease is rare but later-onset inflammatory states are more common (Paediatric Inflammatory Multisystem Syndrome Temporally associated with SARS-CoV-2 (PIMS-TS). Whilst is each presentation mimics other infectious and inflammatory presentations, treatments for early infection (antivirals), severe pulmonary disease, and hyperinflammatory disease (anti-inflammatory drugs) are distinct, and may be mutually harmful.

Overview

RNA signatures associated with COVID-19 can be characterized including viral-driven disease and inflammatory states, in comparison to existing data from comparator febrile illnesses. Minimal signatures that discriminate COVID-19 infectious and inflammatory states from other infectious and inflammatory diseases can be identified. eLAMP assays can be developed for key transcripts that distinguish COVID-19 from other infections, and identify COVID-19 inflammatory syndromes. Variable selection algorithms may be used to identify minimal signatures that discriminate COVID-19 infectious and inflammatory states, and distinguish them from other infectious and inflammatory diseases.

Gene Signatures

A gene signature may comprise one or more genes, selected from the group consisting of: IF144L, EMR1, FAM89A, EB13, IF127L, IFTI1, RSAD2, IFIT3, OTOF, IFIT2, EPSTI1, SERPING1, OAS1, 1F16, HLA-DRB6, HBZ, HS.386275, ElF2AK2, IFIT1L, FCER1A, C210RF7, GYPE, GYPB, HBM, ElF1 AY, LOC649143, HBD, FBXO7, KCNMA1, MERTK, UPB1, PTPN20, TMEM119, SLPI, S100P and P13.

A gene signature may also comprise one or more genes selected from the group consisting of: F144L, EMR1, 1F127, IFIT1, RSAD2, IFIT3, OTOF, IFIT2, ESPTI1, OAS1, 1F16, HS.386275, EIF2AK2, FAM89A, KCNMA1, MERTK, EB13, UPB1, PTPN20, TMEM119, SLPI, S100P and P13.

The Biological Sample and Solution

The sample may be any suitable sample comprising a nucleic acid. For example, the sample may be an environmental sample or a clinical sample. The sample may also be a sample of synthetic DNA (such as gBlocks) or a sample of a plasmid. The plasmid may include a gene or gene fragment of interest.

The environmental sample may be a sample from air, water, animal matter, plant matter or a surface. An environmental sample from water may be salt water, brackish water or fresh water. For example, an environmental sample from salt water may be from an ocean, sea or salt marsh. An environmental sample from brackish water may be from an estuary. An environmental sample from fresh water may be from a natural source such as a puddle, pond, stream, river, lake. An environmental sample from fresh water may also be from a man-made source such as a water supply system, a storage tank, a canal or a reservoir. An environmental sample from animal matter may, for example, be from a dead animal or a biopsy of a live animal. An environmental sample from plant matter may, for example, be from a foodstock, a plant bulb or a plant seed. An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

The clinical sample may be a sample from a patient. The nucleic acid may be a nucleic acid from the patient. The clinical sample may be a sample from a bodily fluid. The clinical sample may be from blood, serum, lymph, urine, faeces, semen, sweat, tears, amniotic fluid, wound exudate or any other bodily fluid or secretion in a state of heath or disease. The clinical sample may be a sample of cells or a cellular sample. The clinical sample may comprise cells. The clinical sample may be a tissue sample. The clinical sample may be a biopsy.

The clinical sample may be from a tumour. The clinical sample may comprise cancer cells. Accordingly, the nucleic acid may be a nucleic acid from a cancer cell.

The sample may be obtained by any suitable method. Accordingly, the method of the invention may comprise a step of obtaining the sample. For example, the environmental air sample may be obtained by impingement in liquids, impaction on is solid surfaces, sedimentation, filtration, centrifugation, electrostatic precipitation, or thermal precipitation. The water sample may be obtained by containment, by using pour plates, spread plates or membrane filtration. The surface sample may be obtained by a sample/rinse method, by direct immersion, by containment, or by replicate organism direct agar contact (RODAC).

The sample from a subject may contain or be suspected of containing a pathogen. Accordingly, the nucleic acid may be a nucleic acid from the pathogen. Alternatively, the nucleic acid may be a nucleic acid from the host.

The method of the invention may be an in vitro method or an ex vivo method.

The pathogen may be a eukaryote, a prokaryote or a virus. The pathogen may be an animal, a plant, a fungus, a protozoan, a chromist, a bacterium or an archaeum.

As used herein, “nucleic acid sequence” may refer to either a double stranded or to a single stranded nucleic acid molecule. The nucleic acid sequence may therefore alternatively be defined as a nucleic acid molecule. The nucleic acid molecule comprises two or more nucleotides. The nucleic acid sequence may be synthetic. The nucleic acid sequence may refer to a nucleic acid sequence that was present in the sample on collection. Alternatively, the nucleic acid sequence may be an amplified nucleic acid sequence or an intermediate in the amplification of a nucleic acid sequence.

As used herein, “anneal”, “annealing”, “hybridise” and “hybridising” refer to complementary sequences of single-stranded regions of a nucleic acid pairing via hydrogen bonds to form a double-stranded polynucleotide. As used herein, “anneal”, “anneals”, “hybridise” and “hybridises” may refer to an active step. Alternatively, as used herein, “anneal”, “anneals”, “hybridise” and “hybridises” may refer to a capacity to anneal or hybridise; for example, that a primer is configured to anneal or hybridise and/or that the primer is complementary to a target. Accordingly, for example, a reference to a primer or a region of a primer which anneals to a nucleic acid sequence or a region of a nucleic acid sequence may in a method of the invention mean either that the annealing is a required step of the method; that the primer or region of the primer is complementary to the nucleic acid sequence or region of the nucleic acid sequence; or that the primer or region of the primer is configured to anneal to the nucleic acid sequence or region of the nucleic acid sequence.

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the nucleic acid primer typically contains 15 to or more nucleotides, although it may contain fewer or more nucleotides. According to the present invention a nucleic acid primer typically contains 13 to 30 or more nucleotides.

The nucleic acid may be isolated, extracted and/or purified from the sample prior to use in the method of the invention. The isolation, extraction and/or purification may be performed by any suitable technique. For example, the nucleic acid isolation, extraction and/or purification may be performed using a nucleic acid isolation kit, a nucleic acid extraction kit or a nucleic acid purification kit, respectively.

The method of the invention may further comprise an initial step of isolating, extracting and/or purifying the nucleic acid from the sample. The method may therefore further comprise isolating the nucleic acid from the sample. The method may further comprise extracting the nucleic acid from the sample. The method may further comprise purifying the nucleic acid from the sample. Alternatively, the method may comprise direct amplification from the sample without an initial step of isolating, extracting and/or purifying the nucleic acid from the sample. Accordingly, the method may comprise lysing cells in the sample or amplifying free circulating DNA.

Following isolation, extraction and/or purification, the nucleic acid may be used immediately or may be stored under suitable conditions prior to use. Accordingly, the method of the invention may further comprise a step of storing the nucleic acid after the extracting step and before the amplifying step.

The step of obtaining the sample and/or the step of isolating, extracting and/or purifying the nucleic acid from the sample may occur in a different location to the subsequent steps of the method. Accordingly, the method may further comprise a step of transporting the sample and/or transporting the nucleic acid.

The method may further comprise diagnosing an infectious disease or a drug resistant infection if the nucleic acid molecule is present.

The method of diagnosis may be an in vitro method or an ex vivo method.

The infectious disease may be selected from the group consisting of Acute Flaccid Myelitis (AFM), Anaplasmosis, Anthrax, Babesiosis, Botulism, Brucellosis, Burkholderia mallei (Glanders), Burkholderia pseudomallei (Melioidosis), Campylobacteriosis (Campylobacter), Carbapenem-resistant Infection (CRE/CRPA), Chancroid, Chikungunya Virus Infection (Chikungunya), Chlamydia, Ciguatera, Clostridium Difficile Infection, Clostridium Perfringens (Epsilon Toxin), Coccidioidomycosis fungal infection (Valley fever), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E. coli infection (E.Coli), Eastern Equine Encephalitis (EEE), Ebola, Hemorrhagic Fever (Ebola), Ehrlichiosis, Encephalitis, Arboviral or parainfectious, Enterovirus Infection, Non-Polio (Non-Polio Enterovirus), Enterovirus Infection, D68 (EV-D68), Giardiasis (Giardia), Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomarivus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LVG), Malaria, Measles, Meningitis, Viral (Meningitis, viral), Meningococcal Disease, Bacterial (Meningitis, bacterial), Middle East Respiratory Syndrome Coronavirus (MERS-CoV), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis, Pthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scabies Infestation (Scabies), Scombroid, Severe Acute Respiratory Syndrome (SARS), Shigellosis gastroenteritis (Shigella), Smallpox, Staphyloccal Infection, Methicillin-resistant (MRSA), Staphylococcal Food Poisoning, Enterotoxin—B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichonosis Infection (Trichinosis), Tuberculosis (TB), Tuberculosis (Latent) (LTBI), Tularemia (Rabbit fever), Typhoid Fever, Group D, Typhus, Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), Zika Virus Infection (Zika) and COVID-19.

Alternatively the infectious or inflammatory condition to be diagnosed may be drawn from a list describing the type of infection or inflammatory condition, rather than the species of pathogen, including but not limited to: viral infections, bacterial infections, gram-positive bacterial infection, gram-negative bacterial infection, mycobacterial infection, autoinflammatory illness, autoimmune illness, Kawasaki disease and PIMS-TS.

A Computing Device and a Computer Readable Medium

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

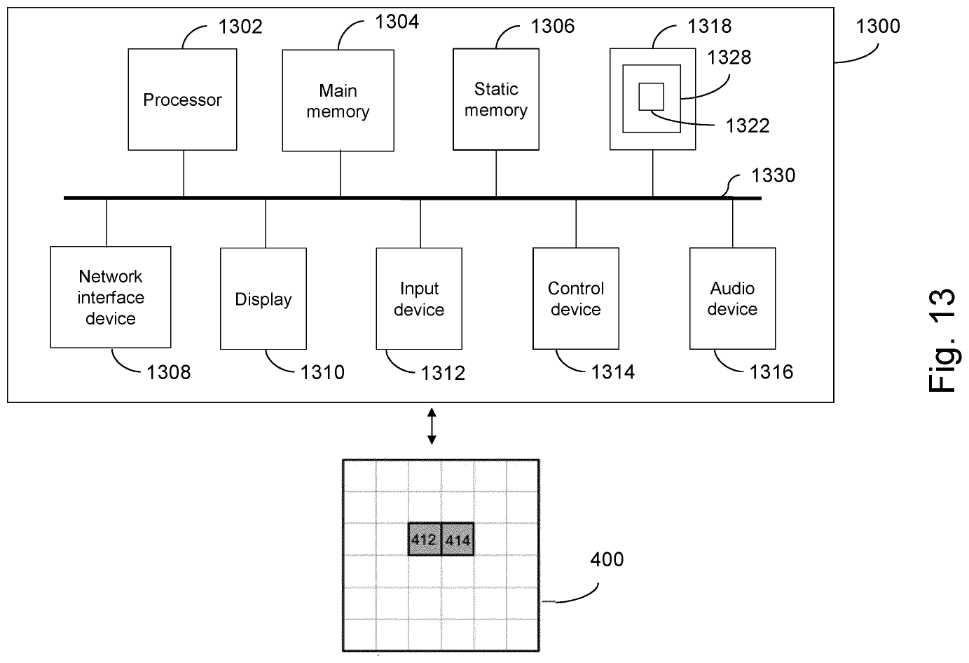

FIG. 13 illustrates a block diagram of one implementation of a computing device 1300 within which a set of instructions, for causing the computing device to perform any one or more of the methodologies discussed herein, may be executed. The computing device is configured to receive signals from an array of electrochemical sensors 400. The depicted array 400 comprises a plurality of sensors, including a first sensor 412 and a second sensor 414. Only two sensors are marked using reference numbers for the purposes of simplicity of illustration. The computing device 1300 may be an integrated chip connected to, i.e. integral with, the array 400, or might be positioned remote from the array 400.

In alternative implementations, the computing device may be connected (e.g., networked) to other machines in a Local Area Network (LAN), an intranet, an extranet, or the Internet. The computing device may operate in the capacity of a server or a client machine in a client-server network environment, or as a peer is machine in a peer-to-peer (or distributed) network environment. The computing device may be a personal computer (PC), an integrated circuit, a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single computing device is illustrated, the term “computing device” shall also be taken to include any collection of machines (e.g., computers) that individually or jointly execute a set (or multiple sets) of instructions to perform any one or more of the methodologies discussed herein.

The example computing device 1300 includes a processing device 1302, a main memory 1304 (e.g., read-only memory (ROM), flash memory, dynamic random access memory (DRAM) such as synchronous DRAM (SDRAM) or Rambus DRAM (RDRAM), etc.), a static memory 1306 (e.g., flash memory, static random access memory (SRAM), etc.), and a secondary memory (e.g., a data storage device 1318), which communicate with each other via a bus 1330.

Processing device 1302 represents one or more general-purpose processors such as a microprocessor, central processing unit, or the like. More particularly, the processing device 1302 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1302 may also be one or more special-purpose processing devices such as an application specific integrated circuit (ASIC), a field programmable gate array (FPGA), a digital signal processor (DSP), network processor, or the like. Processing device 1302 is configured to execute the processing logic (instructions 1322) for performing the operations and steps discussed herein.

The computing device 1300 may further include a network interface device 1308. The computing device 1300 also may include a video display unit 1310 (e.g., a liquid crystal display (LCD) or a cathode ray tube (CRT)), an alphanumeric input device 1312 (e.g., a keyboard or touchscreen), a cursor control device 1314 (e.g., a mouse or touchscreen), and an audio device 1316 (e.g., a speaker).

The data storage device 1318 may include one or more machine-readable storage media (or more specifically one or more non-transitory computer-readable storage media) 1328 on which is stored one or more sets of instructions 1322 embodying any one or more of the methodologies or functions described herein. The instructions 1322 may also reside, completely or at least partially, within the main memory 1304 and/or within the processing device 1302 during execution thereof by the computer system 1300, the main memory 1304 and the processing device 1302 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing “, “enabling”, “maintaining,” “identifying or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Multiple in-house Python (v3.7) scripts were developed to extract and analyze the data using standard data science packages including: NumPy, Pandas and Scikit-Learn.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A processor-implemented method for determining a diagnostic outcome, the method comprising:
receiving signals generated by an array of electro-chemical sensors in solution with a biological sample from a subject, each sensor of the array of electro-chemical sensors configured to generate a signal in response to one or more amplification reactions occurring in the solution, the one or more amplification reactions being indicative of the presence of an RNA host-response;
processing the signals received from the array of electro-chemical sensors;
based on the processing, assigning at least one value to the sample, the at least one value being indicative of a likelihood of the diagnostic outcome; and
based on the at least one assigned value, determining the diagnostic outcome.

2. The method of claim 1, wherein the electro-chemical sensors are any of ion-sensitive field-effect transistor (ISFET) sensors, pH sensors, or chemically sensitive sensors.

3. The method of claim 1, wherein the nucleic acid amplification reaction is an isothermal reaction.

4. The method of claim 3, wherein the amplification reaction is a LAMP reaction.

5. The method of claim 1, wherein the biological sample contains at least one of a DNA, RNA or protein sample.

6. The method of claim 1, wherein the at least one value is assigned to the sample based on a gene expression value and/or time-to-positive.

7. The method of claim 1, wherein the diagnostic outcome is a differentiation between a bacterial and a viral infection.

8. The method of claim 1, wherein the diagnostic outcome is a differentiation between a biological sample from a subject that has an infection and a biological sample from a subject that does not have an infection.

9. (canceled)

10. The method of claim 1, wherein the diagnostic outcome is a differentiation between a biological sample from a subject that is predisposed to an infection and a biological sample from a subject that is not predisposed to an infection.

11. The method of claim 1, wherein the diagnostic outcome is a differentiation between an active form of an infection and a latent form of an infection.

12. The method of claim 1, wherein assigning the at least one value to the sample comprises using a trained classifier.

13. The method of claim 1, wherein the RNA host-response is detected using gene expression levels of a gene signature comprising one or more genes, selected from the group consisting of: IFI44L, EMR1, FAM89A, EBI3, IFI27L, IFTI1, RSAD2, IFIT3, OTOF, IFIT2, EPSTI1, SERPING1, OAS1, IFI6, HLA-DRB6, HBZ, HS.386275, EIF2AK2, IFIT1L, FCER1A, C210RF7, GYPE, GYPB, HBM, EIF1AY, LOC649143, HBD, FBXO7, KCNMA1, MERTK, UPB1, PTPN20, TMEM119, SLPI, S100P and PI3.

14. The method of claim 1, wherein processing the signals comprises clustering the signals received from each sensor of the array of sensors in the time domain.

15. The method of claim 14, wherein each cluster generated by the clustering process comprises a subset of sensors of the array of sensors.

16. The method of claim 1, wherein processing the signals comprises obtaining a feature associated with the amplification curve associated with an amplification reaction, the amplification curve describing a degree of amplification over time; and
wherein the at least one value is assigned based on the feature associated with the amplification curve.

17. The method of claim 16, wherein the feature associated with the amplification curve is one of a time-to-positive and a Ct value.

18. The method of claim 1, wherein processing the signals comprises obtaining a feature associated with a plurality of different amplification curves, each amplification curve being associated with a different amplification reaction and each describing a degree of amplification of a respective amplicon over time; and
wherein the at least one value is assigned based on the plurality of features associated with the amplification curve.

19. A method for determining a diagnostic outcome, the method comprising:
placing a biological sample from a subject in solution with an array of electro-chemical sensors, each sensor of the array of electro-chemical sensors configured to generate a signal in response to one or more amplification reactions occurring in the solution, the one or more amplification reactions being indicative of the presence of an RNA host-response;
processing signals received from the array of electro-chemical sensors;
based on the processing, assigning at least one value to the sample according to the likelihood of a diagnostic outcome; and
based on the at least one assigned value, determining the diagnostic outcome.

20. A system comprising an array of electro-chemical sensors and a processor, each sensor of the array of electro-chemical sensors configured to generate a signal in response to one or more amplification reactions occurring in the solution, the one or more amplification reactions being indicative of the presence of an RNA host-response;
the processor configured to:
process signals received from the array of electro-chemical sensors;
based on the processing, assigning at least one value to the sample, the at least one value being indicative of the likelihood of a diagnostic outcome; and
based on the at least one assigned value, determine a diagnostic outcome.

21. A computer readable medium comprising computer executable instructions which, when executed by a processor, cause the processor to implement the method for determining a diagnostic outcome, the method comprising:
receiving signals generated by an array of electro-chemical sensors in solution with a biological sample from a subject, each sensor of the array of electro-chemical sensors configured to generate a signal in response to one or more amplification reactions occurring in the solution, the one or more amplification reactions being indicative of the presence of an RNA host-response;
processing the signals received from the array of electro-chemical sensors;
based on the processing, assigning at least one value to the sample, the at least one value being indicative of a likelihood of the diagnostic outcome; and
based on the at least one assigned value, determining the diagnostic outcome.
